(19) United States
(12) Patent Application Publication
(10) Pub. No.: US 2017/0173585 A1
(43) Pub. Date: Jun. 22, 2017

(54) POINT OF CARE POLYMERASE CHAIN REACTION DEVICE FOR DISEASE DETECTION

(71) Applicant: ADVANCED THERANOSTICS INC., Oakville (CA)

(72) Inventors: James MAHONY, Oakville (CA); Christopher STONE, Oakville (CA); Hao CHEN, Oakville (CA); Mark COSTA, Oakville (CA); Bernard LIM, Oakville (CA)

(21) Appl. No.: 15/325,660

(22) PCT Filed: Jul. 10, 2015
(86) PCT No.: PCT/CA2015/050648

§ 371 Date: Jan. 11, 2017

Related U.S. Application Data
(60) Provisional application No. 62/023,468, filed on Jul. 11, 2014.

(51) Int. Cl. B01L 7/00; C12Q 1/68; B01L 3/00

(52) U.S. Cl. CPC B01L 7/52; B01L 3/502715; B01L 3/5029; C12Q 1/6825; B01L 2200/147; B01L 2300/024; B01L 2300/027; B01L 2300/0663; B01L 2300/0816; B01L 2300/1827; B01L 2400/0487; B01L 2300/023

(57) ABSTRACT

A point-of-care device for detecting a target nucleic acid is provided. The device comprises: an extraction chamber adapted to receive a biological sample, wherein said extraction chamber comprises means to extract and lyse the sample to release nucleic acid; a first amplification chamber in communication with the extraction chamber, wherein said amplification chamber comprises means to trigger nucleic acid amplification of a target nucleic acid sequence to occur; and a detection chamber in communication with the amplification chamber, wherein said detection chamber comprises means to detectably label the target nucleic acid and means to detect a signal associated with labeled target nucleic acid.

### FIELD OF THE INVENTION

The present invention pertains to the field of point-of-need (PON) or point-of-care (POC) diagnostic devices, for example, for use in the detection of infectious diseases.

### BACKGROUND OF THE INVENTION

There has been a shift away from traditional testing methods for infectious diseases, such as culture and antigen detection, towards more sensitive nucleic acid amplification tests (herein referred to as NAAT). Polymerase chain reaction (PCR) amplification has provided laboratories with sensitive and specific tools to detect infectious diseases, and has been adopted by clinical laboratories around the world.

Current molecular diagnostic tools are limited by substantial “off-chip” clinical sample preparation time and the requirement for skilled technicians. This limits the application of molecular diagnostics in an at-home or resource-poor setting. Typical diagnostic tests may be completed within a time period ranging from 3 hours to 5 days. However, this does not provide useful diagnostic information in certain circumstances.

Using microfluidic devices to miniaturize diagnostic assays into a “lab on a chip” format has gained much attention in the last decade. Microfluidic devices are typically small and require very low sample volumes, which is conducive to molecular diagnostics. This technology may be useful to construct POC diagnostic tools for use, for example, at the bedside and to provide rapid diagnostic results. However, the cost of a POC device is important and should be as low as possible, especially for use in resource-poor settings. At this time, complicated and expensive sample preparation and DNA detection technologies have prevented the construction of an inexpensive, fully disposable POC device.

Devices have been developed which permit isolation of infectious particles (virus, bacteria or fungi) from a clinical sample (blood, urine, nasopharyngeal swab, fecal material) in an automated manner “on-chip”, or without the need for human intervention. For example, WO110019A1 discloses a microfluidic platform that can bind pathogens or nucleic acid to magnetic beads, and subsequently move them to a secondary chamber for detection. In addition, WO122564A2 discloses a device for the release of intracellular contents from pathogens and subsequent transport of a portion of the contents for detection. US 2008/0299648A1 discloses a self-contained diagnostic kit that includes a sample collection element and an immunochromatography test strip. U.S. Pat. No. 8,574,923B2 discloses a sample preparation device that specifically binds nucleic acids using a monolith absorbent or using sample filters to bind any analytes of interest and lyse cell membranes. WO2012/013733A1 discloses a device for generic sample preparation to isolate nucleic acids from a variety of liquid matrices for diagnostic purposes. Finally, WO2013/158686 A1 discloses a nucleic acid sample preparation device that requires minimal hands on time and can purify nucleic acids from various cellular mixtures.

In the detection of infectious disease, infectious particles are first isolated and then must be lysed to release intracellular contents, including DNA, RNA, and protein. While several methods have been characterized for releasing nucleic acids and proteins from cells, their integration into a diagnostic POC platform significantly increases the complexity of the device. For mechanical lysis, motor elements are required which can increase both the cost and complexity of the device. For chemical lysis, it is difficult to administer the correct amount of lysis reagent and subsequently remove the reagent before analysis downstream. NAAT techniques are particularly sensitive to chemical contamination and all lysis chemicals must be removed before next steps, including enzymatic amplification and detection.

Polymerase chain reaction (PCR) is a common technique used to amplify pathogenic DNA sequences to high concentrations using a combination of thermostable DNA polymerase and thermocycling. However, PCR typically requires thermocycling between ˜50° C. and 95° C. for amplification to occur, and integration of thermocycling into a POC diagnostic platform would increase both the complexity and cost of developing a POC molecular diagnostic device.

The use of disposable systems for POC diagnostics is appealing for at-home testing, resource-poor settings or community hospitals without access to a central laboratory, but auxiliary systems required for read-out are typically expensive and dedicated, which limit their disposability. This can also result in an expensive initial capital investment for a stand-alone unit or reader.

Thus, it would be desirable to develop a portable and disposable diagnostic point-of-care device.

### SUMMARY OF THE INVENTION

A point-of-care device has now been developed which is adapted to receive a raw clinical sample (e.g. blood, urine, fecal material, nasopharyngeal swab and the like), release pathogen intracellular contents, and amplify and detect pathogen nucleic acid.

Accordingly, a point-of-care device is provided comprising:
an extraction chamber adapted to receive a biological sample, wherein said extraction chamber comprises means to extract and lyse the sample to release nucleic acid;
a first amplification chamber in communication with the extraction chamber, wherein said amplification chamber comprises means to trigger nucleic acid amplification of a target nucleic acid sequence to occur; and
a detection chamber in communication with the amplification chamber, wherein said detection chamber comprises means to detectably label the target nucleic acid and means to detect a signal associated with labeled target nucleic acid.

In another aspect of the invention, a method of detecting target nucleic acid in a biological sample is provided comprising:
collecting a biological sample on a swab and inserting the swab in a contained extraction chamber adapted to extract the sample from the swab and lyse pathogen to release nucleic acid;
transferring a portion of the released nucleic acid to a nucleic acid amplification chamber connected to the extraction chamber and exposing the nucleic acid to reagents to induce nucleic acid amplification;
labeling and detecting target nucleic acid in a detection chamber connected to the amplification chamber.

These and other aspects of the invention will become apparent by reference to the figures and description that follow.

### BRIEF DESCRIPTION OF FIGURES

FIG. 1 is a block diagram of an embodiment of the invention (A), and a schematic of a POC device in accordance with an embodiment of the invention (B) including various views;

FIG. 2 graphically illustrates that a self-regulated heater could heat 250 μl of water to 95° C. at different resistances (3.8 ohm, 3.5 ohm, and 3.4 ohm) for 20 minutes by applying a voltage;

FIG. 3 graphically illustrates that a self-regulated heater could heat 250 μl of water to 62° C. at a resistance of 8.6 ohms by applying a voltage;

FIG. 4 graphically illustrates detection of S. agalactiae following amplification, using a fluorescent dye (SYBR Green);

FIG. 5 illustrates analysis of a colour change before and after amplification using Quant-iT PicoGreen DNA binding dye;

FIG. 6 graphically illustrates electrochemical detection of amplified S. agalactiae DNA using methylene blue at 2 concentrations and measuring peak anodic current;

FIG. 7 is a block diagram showing an exemplary computer system which may be used to implement aspects of the present technology; and

FIG. 8 is a block diagram showing an exemplary smartphone which may be used to implement aspects of the present technology

### DETAILED DESCRIPTION

A point-of-care device for use in the detection of a target nucleic acid is provided. The device comprises an extraction chamber adapted to receive a biological sample and lyse the sample to release nucleic acid; a first amplification chamber in communication with the extraction chamber which receives sample from the extraction chamber and comprises means to amplify a target nucleic acid in the sample; and a detection chamber in communication with the amplification chamber comprising means to label the target nucleic acid for detection and means to detect the label. As used herein, the term “extraction chamber” refers to a chamber in which both sample extraction and lysis occurs.

The biological sample may be obtained using any appropriate vehicle for use to transfer the sample into the extraction chamber of the device. In one embodiment of the invention, a swab is used to collect a nucleic acid-containing biological or clinical sample (e.g. blood, urine, nasopharyngeal swab, fecal sample, vaginal swab, tears, fluid excreted at wound sites or sites of inflammation, or any other clinical material that is nucleic acid-containing). As one of skill in the art will appreciate, the biological sample may be obtained by means other than a swab, e.g. by a syringe, or a collection vessel, into which the swab may be dipped. The sample-containing swab is placed in a swab-accepting opening in the extraction chamber of the device. The swab used may be a standard swab, or a swab designed specifically for the device. For example, the swab may sized to fit within the extraction chamber to permit sealing of the opening of the chamber with a cap or lid. When a standard swab is used, the swab shaft may be broken at the opening of the chamber to permit sealing of the chamber opening with a lid. The swab may have a smaller head conducive for collecting certain samples such as vaginal and nasopharyngeal samples. Alternatively, the swab may include a plug along its shaft which functions to seal the opening of the extraction chamber and prevent leakage from the device. Once the extraction chamber is closed, it forms an enclosed contained environment within the device. Other appropriate vehicles for sample transfer include a stick, a foam-tipped shaft, or other vehicle capable of adsorbing the biological sample for transfer to the device.

The extraction chamber comprises means to enable sample extraction, as required, and lysis to release nucleic acid from the sample. Thus, the extraction chamber either contains or has access to a lysis solution suitable for extraction of sample from the delivery vehicle and lysis of the sample, for example, a phosphate buffered saline solution, water, a 0.1% Triton-X100 solution, a 0.1% SDS solution, other suitable detergents for extraction and lysing, or a combination of any of these. In one embodiment, the extraction chamber includes an amount of the lysis solution suitable for extraction and lysis, e.g. a volume of about 0.2-0.5 mL of lysis solution, to immerse the sample-containing vehicle and facilitate sample extraction/lysis therefrom. In this case, the extraction chamber is provided with a one-way point of entry (at or adjacent to the opening of the device), e.g. a membrane, which permits input of the sample into the extraction chamber via a vehicle (e.g. swab or the like), but prevents leakage of lysis solution from the extraction chamber. In another embodiment, the extraction chamber is in communication with a buffer-releasing means which functions to release extraction and lysis solution into the extraction chamber on entry of the sample into the extraction chamber. For example, the lysis solution may be contained in a pouch, blister pack or other reservoir, either within the extraction chamber or adjacent to the extraction chamber, which is activated to release solution into the extraction chamber on entry of the sample. In this regard, the pouch or blister-pack may be pierced by the sample-containing vehicle (e.g. swab) on entry, pierced by means within the chamber on sealing of the chamber or closure of the lid, or burst by pressure within the chamber on sealing of the chamber. An adjacent reservoir may be caused to release lysis solution into the extraction chamber by similar piercing or bursting of a membrane connecting the reservoir to the extraction chamber. As will be appreciated by one of skill in the art, other means of releasing lysis solution from a pouch or reservoir may also be utilized.

On sealing of the extraction chamber, for example, by closure of the lid to the opening of the extraction chamber, the device is activated by completion of one or more circuits as will be described. Thus, heater(s), pump(s), and other electrical parts (as described) are appropriately powered by connection to a control unit, including a battery, either on-board or off-board (via connection to an external power source), via any appropriate adaptors (DC adaptor) and/or converters (D/A converter). The connection may be a standard electrical connection (DC adaptor) to a power source (e.g. battery), or the connection may be via a port (e.g. USB) to an external power source such as a processing device, e.g. computer, cell phone, tablet or other external device. Thus, the device may be provided with means to connect to a power source.

The extraction chamber comprises means to facilitate sample extraction and lysis to release nucleic acid therefrom. In one embodiment, a heating means, e.g. a self-regulating heater, is used to heat the extraction chamber to a temperature suitable to facilitate sample extraction and lysis, e.g. a temperature between about 88° C.-100° C. for a sufficient time period, e.g. at least about 2-3 minutes. Self-regulating heaters may include, but are not limited to, PTC (Positive Temperature Coefficient) ceramic heaters, evaporation temperature control heaters, or heat-sink temperature control heaters. The heater is activated when the lid of the extraction chamber is closed, thereby completing a circuit which connects the heater to a power supply, e.g. battery.

Alternatively, the extraction chamber may comprise means to generate a mechanical force, e.g. a small motor, to facilitate release of sample from the swab and lysis of any pathogen present. The motor may be combined with the use of glass or ceramic beads placed within the extraction chamber to accelerate lysis. The motor may be situated at the base of the extraction chamber, and is powered in the manner described for the heater. This embodiment may or may not additionally include a heater to facilitate lysis.

In certain samples, for example nasopharyngeal samples, pathogens can be lysed with heat and nucleic acid can be directly amplified without nucleic acid purification.

In other samples, for example blood or urine which contain nucleic acid amplification inhibitors (such as bile salts, heme, proteinases, urea, or hemoglobin); purification of nucleic acid within the extraction chamber may be required. In this case, the extraction chamber may be coated with immobilized oligonucleotide capture probes that are homologous to the target nucleic acid (e.g. in an amount of about 10⁴ to 10⁵ copies), or is coated with target nucleic acid-specific oligonucleotide probes for a target pathogen sequence (for example, such as those exemplified in Table 1). The device May optionally include a positively charged electrode within the extraction chamber which is powered or activated on closure of the lid of the device, to facilitate nucleic acid binding onto the capture probes. Once nucleic acid is bound to the probes, e.g. within a short period of time such as about 2 minutes, unbound contaminants may be removed from the extraction chamber. Contaminant removal may be accomplished by aspiration, or pumping, into a waste chamber connected to the extraction chamber, or contaminants may be washed into the waste chamber with buffer released from a secondary buffer chamber. This step removes potential amplification inhibitors and concentrates the nucleic acid, e.g. DNA or RNA, for amplification. This nucleic acid binding step is performed between room temperature and 35° C., without the need for a heater. Following binding, the means used to remove contaminants from the extraction chamber, e.g. micro-pump and/or buffer-releasing means, is activated as previously described, and may utilize a timer so that waste removal occurs following capture of all or a sufficient quantity of nucleic acid. Nucleic acid may then subsequently be eluted as previously described.

Following lysis and nucleic acid release, a measured volume (e.g. about 5-10 μl) of this lysed material is transferred, for example by a micro-pump, to the amplification chamber via a channel, such as a microfluidic channel. The micro-pump may be powered by an on-board power source, such as a battery, or through connection to an external power source, as described. The pump is activated at the appropriate time, e.g. once sample extraction and lysis is complete, to transfer lysed material to the amplification chamber. In one embodiment, activation of the pump is delayed by a timer, connected to the power supply, to ensure that the sample undergoes extraction and lysis, and thereby to prevent transfer of unlysed material to the amplification chamber. Regarding transfer of a measure volume of lysed material, this may be controlled by a microcontroller. Alternatively, the volume of lysed material transferred into the amplification chamber may be controlled by the size of the amplification chamber (e.g. sized to contain a sufficient amount of amplification mixture and the desired amount of lysed material). In this case, the entrance to the amplification chamber may be covered by a hydrophobic membrane that permits output of gases from within the amplification chamber as it is filled, and input of liquid into the chamber until the chamber is filled.

The amplification chamber contains an amplification mixture which enables nucleic acid amplification to occur. The amplification mixture present in the amplification chamber may be lyophilized (optionally stabilized with pullanin or trehalose), in which case about 25 to 50 μl of lysed solution is added to the well. The amplification may also be in liquid form, in which case about 5 to 10 μl of lysed solution is added to the well. The amplification mixture contains oligonucleotide primers for amplification of target nucleic acid sequences (e.g. about 0.2 to 1.8 μM), a strand-displacement DNA polymerase, such as Taq polymerase (e.g. about 8 units), deoxynucleoside triphosphates or dNTPs (e.g. about 15 to 30 mM of each), buffer (e.g. about 1× final concentration), cation such as magnesium (e.g. about 2.0 to 8.0 mM). When the target nucleic acid is RNA, the amplification mixture additionally includes a reverse transcriptase. As one of skill in the art will appreciate, the oligonucleotide primers are selected to amplify a particular target DNA sequence from a target microorganism. Thus, to amplify a target sequence, primers (e.g. comprising from about 10 up to about 100 bases) which are complementary to a DNA sequence within the target microorganism are utilized. As one of skill in the art will appreciate, the number of oligonucleotide primers in the amplification mixture will vary with the amplification technique used, and primers in both the 5′-3′ orientation as well as orientation may be used. In one embodiment, the target microorganism is a pathogenic organism such as, but not limited to, Escherichia coli, Listeria monocytogenes, Clostridium Mycoplasma pneumonia, Chlamydia pneumoniae, Chlamydia trachomatis, Legionella pneumophilia, Neisseria gonorrhea, Streptococcus sp. including Group B streptococcal infection, Herpes, papillomavirus, Staphylococcus sp. including Methicillin-resistant Staphylococcus aureus (MRSA), Influenza virus, Respiratory Syncytial Virus, Norovirus, West Nile Virus, Dengue Virus, SARS Co-V, Ebola virus, Lassa fever virus, Tuberculosis, HIV, Middle East respiratory syndrome coronavirus, and Chikungunya virus. Examples of primers used to amplify some of these pathogens can be found in Table 1.

In one embodiment, amplification is accomplished by an isothermal amplification technique, including but not limited to, loop-mediated isothermal amplification (LAMP), cross-priming amplification (CPA), recombinase polymerase amplification (RPA), rolling circle amplification (RCA), helicase-dependent amplification (HDA), single-mediated amplification of RNA technology (SMART), nicking enzyme-mediated amplification (NEMA), isothermal chain amplification (ICA), Smart amplification (Smart-AMP), exponential amplification reaction (EXPAR), or ramification amplification (RAM). During amplification, the chamber is heated to a temperature suitable for amplification, e.g. a temperature between about 58° C. to 66° C., for a sufficient period of time, e.g. about 15-20 minutes, using a heater, such as a self-regulating heater. The heater is activated at the appropriate time, e.g. on or slightly prior to transfer of lysed material into the amplification chamber. In one embodiment, activation of the heater is delayed by a timer, connected to the power supply, to prevent premature heating within the amplification chamber.

In another embodiment, amplification may be accomplished by pH cycling-dependent amplification. In this case, the pH of the solution is cycled, for example from about pH 3 to about pH 8, to denature and renature the nucleic acid, thereby allowing polymerase access and subsequent amplification. For example, the amplification chamber may comprise a hydrogen-loaded plate dividing the amplification chamber into two sections. Electric field generated by two electrodes on either side of the plate pull hydrogen ions back and forth, cycling the pH. This is controlled by an electrical or mechanical timer to activate the electrodes on either side of the plate.

Electrical-Field amplification (EFA) may also be used for nucleic acid amplification whereby an electric field is applied to denature the DNA and thereby to allow access by the polymerase without the need for thermocycling. In this case, an electric field in the range of about 0.01 to 0.1 mV is generated by applying a voltage across electrodes located at opposite sides of the amplification chamber using a suitable power source. The voltage is activated and deactivated (to cause denaturing followed by renaturing and amplification) at specific intervals of between 10 and 20 seconds using a mechanical or electrical timer.

In another embodiment, the device is adapted for use to perform thermal cycling PCR amplification in the amplification chamber. For this approach, the self-regulated heater within the amplification chamber is activated and deactivated at specific time intervals (e.g. 20-40 s) using a mechanical or electric timer to cause heating up to a denaturing temperature, e.g. 94-96° C., and cooling to an annealing/amplification temperature, e.g. about 70° C. Temperatures are monitored with a thermistor connected to the microprocessor. As one of skill in the art will appreciate, the temperatures utilized and their time intervals may vary with the polymerase used, the concentration of divalent ions and dNTPs in the reaction, and the melting temperature of the primers.

The device may optionally include a second (parallel) amplification chamber to amplify nucleic acid sequences within the sample to serve as an amplification control. Thus, the second amplification chamber will include amplification mixture, along with oligonucleotide primers directed to the control nucleic acid sequence, such that when the device is activated, amplification of the control sequence occurs. While any suitable control sequence may be used, as one of skill in the art will appreciate, examples of control sequences include human genes such as human β-actin, as well as nucleic acid sequence from commensal bacteria such as Streptococcus anginosus or Staphylococcus epidermidis. Thus, amplification of the control sequence will confirm that the sample was properly obtained, extracted and lysed, and that amplification properly occurred within the device, and that lack of a signal for the target microorganism is due to lack of target sequence within the sample as opposed to malfunction of the device.

Following amplification, the presence of target nucleic acid may be detected using a variety of methods including but not limited to: electrochemical detection, lateral flow-based detection, fluorescence detection, or colorimetric detection. This step is completed either within the amplification chamber or in a separate detection chamber, whereby a portion of the fluid is transported to a detection chamber using a micropump powered as previously described and using a timer to delay transport of fluid into the detection chamber until amplification is complete. Detection of DNA amplification may be performed directly in the amplification chamber in the presence of a detection sensor such as electrochemical detectors (e.g. potentiostat), light detectors (e.g. photodiode, fluorometer), colorimetric detector (e.g. light meter), and the like. Alternatively, detection may be performed in a separate detection chamber including a detection sensor. For colorimetric detection, the detection sensor may be a window that permits viewing of a colour change within the detection chamber.

To enable detection of target nucleic acid, it is labeled with a DNA-binding detectable label, such as fluorescent, chemiluminescent, and chromogenic labels, and electrochemically detectable labels. Examples of suitable DNA-binding detectable labels include methylene blue dye, leucocrystal violet, Quant-iT PicoGreen, or cyanin DNA binding dyes.

In one embodiment, a DNA-binding detectable label is present in the amplification mix and binds to DNA as the DNA concentration increases by intercalating into the double helix of DNA. The amount of DNA-binding detectable label added to the amplification chamber is an amount in the range of about 5 to 10 The labeled DNA, e.g. DNA/methylene blue complex, migrates differently to unbound label, e.g. methylene blue alone, in the presence of an electric field, which is used as a measure of DNA concentration. The electric field is provided by electrodes present within the amplification chamber and submerged in the sample. The electrodes are connected to a potentiostat which provides the voltage, after which peak anodic current is measured and relayed to an on-board or external microprocessor.

DNA amplification may be monitored using a DNA binding fluorescent dye. Examples of suitable DNA-binding fluorescent dyes include SYBR green, CYTO9, hydroxynapthol blue, or Quant-iT PicoGreen, either in the amplification chamber or a separate detection chamber. The amount of DNA-binding fluorescent dye used for detection is an amount in the range of about 5 to 10 μl. The device may be equipped with a fluorescent detector connected to the amplification or detection chamber, and a processing unit to provide a processed output. Alternatively, the signal from the detector may be transferred to an external processing unit to provide a processed output.

Amplification of DNA may be detected colorimetrically using gold nano-particles, DNAzymes, or DNA binding dyes as described above, that trigger a colour change in the presence of DNA. This colour change may be detected manually (by eye) through a window which permits viewing into the detection chamber, using an on-board colorimetric detector connected to the detection or amplification chamber, or using an off-board detection means to which the detection chamber is connected as described, e.g. to analyze a color image of the solution following reaction with a colorimetric label by analysis of the colour change, such as RGB analysis. This may also be accomplished by shining a matching colour light onto the final amplified reaction mixture and monitoring the colour intensity as an output with a light meter.

Amplified DNA may be detected using lateral flow assay in the amplification chamber or a detection chamber. Multiple arrangements for detection using lateral flow assay are possible. In one embodiment, the amplified DNA is tagged with a ligand (e.g. biotin or another ligand) and labeled-oligonucleotide primers to the target nucleic acid (e.g. labeled with a fluoroscein label such as 6-carboxyfluorescein or fluorescein isothiocyanate (FITC), or another label), subsequently complexed using a binder to the ligand (e.g. avidin or streptavidin) and captured by immobilized antibody (e.g. anti-FITC antibody). As described above, the captured amplified DNA is then detected based on the label used, e.g. by a fluorescent or colorimetric signal, as described above.

In another embodiment of the device, detection is performed in a separate detection chamber containing a DNA binding dye which cannot be present during amplification, e.g. Herseh dye. The amount of DNA-binding dye used for detection is an amount in the range of about 5 to 10 μl. DNA binding is monitored by electrochemical detection in the presence of an electric field as described for methylene blue DNA binding.

The detection chamber may optionally be a vial removably connected to the outside of the device that is amenable to subsequent analysis.

In another embodiment, the device may be adapted such that lysis, amplification and detection is performed in a single chamber. To accomplish this, the vehicle containing the clinical sample is immersed into a single chamber containing amplification mixture (e.g. heat-stable DNA polymerase and/or reverse transcriptase for RNA targets, magnesium, nucleotides, nucleic acid primers for a specific target, and a DNA binding dye). The chamber including a self-regulating heater is activated by a timer to heat to a temperature of about 95° C. to lyse pathogens. Alternatively, free DNA/RNA is amplified without the need for heating to 95° C. In this case, the heating step can be omitted. The heater is then deactivate to permit cooling of the entire sample to a temperature between about 50-70° C. for DNA amplification. Following DNA amplification, the DNA binding dye will react with any amplified DNA and result in a colour change within the single chamber which may be detected as previously described.

In a further embodiment, the device may be adapted such that lysis and amplification occur in a single chamber, while detection occurs in a separate detection chamber. In this case, the lysis-amplification chamber includes a self-regulated heater which is activated and deactivated at specific time intervals using a mechanical or electric timer to allow for heating up to a denaturing temperature, e.g. 95° C. and cooling to an amplification temperature, e.g. about 70° C. Amplified DNA may then be transported, for example via a pump, into a separate detection chamber for detection as previously described.

In another embodiment, the device may be adapted to detect two or more microorganisms, e.g. two or more pathogens. In this embodiment, the device may comprise two or more amplification chambers, each adapted to amplify the nucleic acid of a different target organism. Thus, each of the amplification chambers of this embodiment of the device will include an amplification mixture targeted to a different microorganism, including oligonucleotide primers for the targeted microorganism. For example, the first amplification chamber may include oligonucleotide primers for Escherichia coli, while the second amplification chamber includes oligonucleotide primers for Listeria monocytogenes, and the third amplification chamber includes oligonucleotide primers for S. aureus. In another example, the device may include amplification chambers targeted to various skin infections, such as Herpes, papillomavirus and S. aureus. The device may be adapted for use in a third world country, and include amplification chambers each adapted to identify relevant target organisms such as, but not limited to, Dengue Virus, SARS Co-V, Ebola virus, Lassa fever virus, Tuberculosis and/or HIV.

The detection sensor of the device may be adapted for connection to a signal processing unit operable to receive the signal provided by the detection sensor and to translate the signal into a desired output. Thus, the signal processing unit is operable to digitize the output provided by the detection sensor, if required, into a recordable output which may be presented, for example, on a display, e.g. monitor or the like. For example, in one embodiment, the results of the diagnostic assay can be transmitted to an on-board intelligent reader for the user to view results. The signal processing unit may be included within the device in the form of a microprocessor (e.g. digital signal processor) including any required convertors to translate the output from the detection sensor (analog to digital convertor), or in the form of a digital acquisition board to digitize the signal from the detection sensor. Alternatively, the signal processing unit may be an external processing system. In such an embodiment, the device is equipped with a port for communication with an external processing system. The port may be a physical port (e.g. a USB port) which may function to transfer power to the device from the external processing system, and to transfer output from the detection sensor to the external processing system for signal processing. Alternatively, the port may be a wireless communication port, for example using the WiFi or Bluetooth protocols, which functions to transfer output from the detection sensor to the external processing system for signal processing. Examples of external processing systems include personal computers, personal digital assistants, networked mobile wireless telecommunication computing devices such as smartphones, and content players.

Aspects of the present technology used to implement the signal processing unit may be embodied within a system, a method, a computer program product or any combination thereof. The computer program product may include a computer readable storage medium or media having computer readable program instructions thereon for causing a processor to carry out aspects of the present technology. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing.

A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present technology may be assembler instructions, instruction-set-architecture (ISA) instructions, machine instructions, machine dependent instructions, microcode, firmware instructions, state-setting data, or either source code or object code written in any combination of one or more programming languages, including an object′ oriented programming language or a conventional procedural programming language. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to implement aspects of the present technology.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks. The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

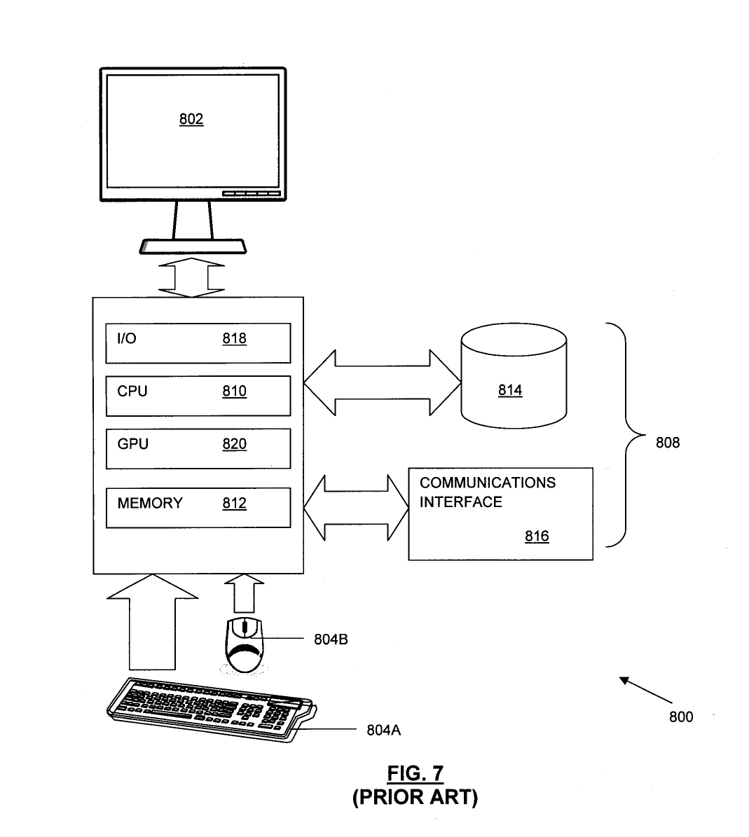

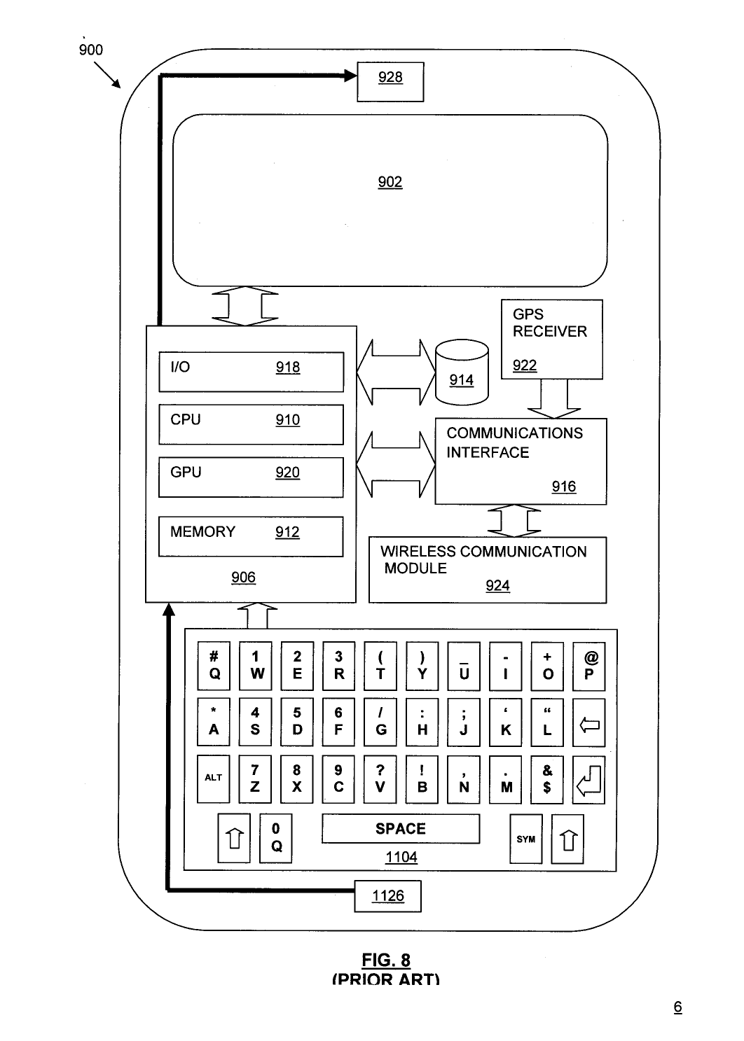

An illustrative computer system in respect of which aspects of the technology herein described may be implemented (e.g. which may function as a signal processing unit) is presented as a block diagram in FIG. 7. The illustrative computer system is denoted generally by reference numeral 800 and includes a display 802, input devices in the form of keyboard 804A and pointing device 804B, computer 806 and external devices 808. While pointing device 804B is depicted as a mouse, it will be appreciated that other types of pointing device may also be used.

The computer 806 may contain one or more processors or microprocessors, such as a central processing unit (CPU) 810. The CPU 810 performs arithmetic calculations and control functions to execute software stored in an internal memory 812, preferably random access memory (RAM) and/or read only memory (ROM), and possibly additional memory 814. The additional memory 814 may include, for example, mass memory storage, hard disk drives, optical disk drives (including CD and DVD drives), magnetic disk drives, magnetic tape drives (including LTO, DLT, DAT and DCC), flash drives, program cartridges and cartridge interfaces such as those found in video game devices, removable memory chips such as EPROM or PROM, emerging storage media, such as holographic storage, or similar storage media as known in the art. This additional memory 814 may be physically internal to the computer 806, or external as shown in FIG. 7, or both.

The computer system 800 may also include other similar means for allowing computer programs or other instructions to be loaded. Such means can include, for example, a communications interface 816 which allows software and data to be transferred between the computer system 800 and external systems and networks. Examples of communications interface 816 can include a modem, a network interface such as an Ethernet card, a wireless communication interface, or a serial or parallel communications port. Software and data transferred via communications interface 816 are in the form of signals which can be electronic, acoustic, electromagnetic, optical or other signals capable of being received by communications interface 816. Multiple interfaces, of course, can be provided on a single computer system 800.

Input and output to and from the computer 806 is administered by the input/output (I/O) interface 818. This I/O interface 818 administers control of the display 802, keyboard 804A, external devices 808 and other such components of the computer system 800. The computer 806 also includes a graphical processing unit (GPU) 820. The latter may also be used for computational purposes as an adjunct to, or instead of, the (CPU) 810, for mathematical calculations.

The various components of the computer system 800 are coupled to one another either directly or by coupling to suitable buses.

In another embodiment, the results can be interpreted using a networked mobile wireless telecommunication computing device such as a smartphone programmed with a specific application for interpreting results. FIG. 9 shows an exemplary networked mobile wireless telecommunication computing device in the form of a smartphone 900; the smartphone 900 which may function as a signal processing unit. The smartphone 900 includes a display 902, an input device in the form of keyboard 904 and an onboard computer system 906. The display 902 may be a touchscreen display and thereby serve as an additional input device, or as an alternative to the keyboard 904. The onboard computer system 906 comprises a central processing unit (CPU) 910 having one or more processors or microprocessors for performing arithmetic calculations and control functions to execute software stored in an internal memory 912, preferably random access memory (RAM) and/or read only memory (ROM) is coupled to additional memory 914 which will typically comprise flash memory, which may be integrated into the smartphone 900 or may comprise a removable flash card, or both. The smartphone 900 also includes a communications interface 916 which allows software and data to be transferred between the smartphone 900 and external systems and networks. The communications interface 916 is coupled to one or more wireless communication modules 924, which will typically comprise a wireless radio for connecting to one or more of a cellular network, a wireless digital network or a Wi-Fi network. The communications interface 916 will also typically enable a wired connection of the smartphone 900 to an external computer system. A microphone 926 and speaker 928 are coupled to the onboard computer system 906 to support the telephone functions managed by the onboard computer system 906, and UPS receiver hardware 922 may also be coupled to the communications interface 916 to support navigation operations by the onboard computer system 906. Input and output to and from the onboard computer system 906 is administered by the input/output (I/O) interface 918, which administers control of the display 902, keyboard 904, microphone 926 and speaker 928. The onboard computer system 906 may also include a separate graphical processing unit (GPU) 920. The various components are coupled to one another either directly or by coupling to suitable buses.

The term “computer system” and related terms, as used herein, are not limited to any particular type of computer system and encompasses servers, desktop computers, laptop computers, networked mobile wireless telecommunication computing devices such as smartphones, tablet computers, as well as other types of computer systems.

Thus, computer readable program code for implementing aspects of the technology described herein may be contained or stored in the memory 912 of the onboard computer system 906 of the smartphone 900 or the memory 812 of the computer 806, or on a computer usable or computer readable medium external to the onboard computer system 906 of the smartphone 900 or the computer 906, or on any combination thereof.

Thus, in one embodiment of the device, diagnostic results are sent to a smartphone, laptop or any suitable computing device using either a wired or wireless connection. The computing device will include software that can interpret and present the diagnostic information for the user. In addition, the PON diagnostic platform can be powered using an on-board battery source, through a DC power adaptor, or by a smartphone, computer or other device through a USB or other suitable connection.

In another embodiment of the device, an on-board reader will be available that will directly analyze/interpret/display the POCT result for the user.

In one embodiment, each device will include a Radio Frequency Identification (RFID) tag to allow for tracking and data analysis.

### Potential Applications

A disposable, inexpensive POC device is provided which has applications across a broad range of disciplines and sectors. At the outset, the device is advantageously made to be disposable to keep manufacturing costs manageable, to avoid the necessity of cleaning the device after use, and to avoid spread of possible pathogenic infection by further handling of a used device. Following use, the device may be disposed of using protocol established to avoid spread of infection.

One use of this POC device is the rapid detection of pathogenic microorganisms. For example, the POC device may be used to detect pathogenic microorganisms such as, but not limited to, Escherichia coli, Listeria monocytogenes, Clostridium difficile, Mycoplasma pneumonia, Chlamydia pneumoniae, Chlamydia trachomatis, Legionella pneumophilia, Neisseria gonorrhea, Streptococcus, Staphylococcus, Influenza virus, Respiratory Syncytial Virus, Norovirus, West Nile Virus, Dengue Virus, SARS Co-V, Ebola virus, Lassa fever virus, Tuberculosis, HIV or Middle East respiratory syndrome coronavirus.

Thus, the present POC device is particularly useful to detect infectious disease in resource-poor settings without access to a central laboratory for molecular testing. For example, this device can be used to detect infectious disease in Africa, e.g. diarrheal disease using rectal swabs, or HIV. The POC device is useful for testing surfaces in food-processing plants for Listeria or E. coli contamination. The POC device can be used for real-time contamination monitoring in food-processing plants and to ensure sterilization during cleaning processes, and to prevent food-associated outbreak of gastrointestinal diseases. Another application of the POC device is the detection of disease in animals, such as porcine-respiratory virus in pigs by veterinarians, which severely affects the porcine industry. This device can also be used to monitor nosocomial (hospital-acquired) infections in nursing and old-age homes, or upon admission to the hospital. Another application of this device is at-home testing for sexually transmitted infections including Chlamydia. Women can take a vaginal swab, which is highly sensitive for Chlamydia detection, and use the POC device for analysis. This device can also be used for real-time surveillance and outbreak control in large populations. In addition, it can be used to respiratory virus testing for passengers embarking or disembarking planes.

Embodiments of the invention are described in the following specific example, which is not to be construed as limiting.

### Example 1—A PON Device for Pathogen Detection

### Device Design

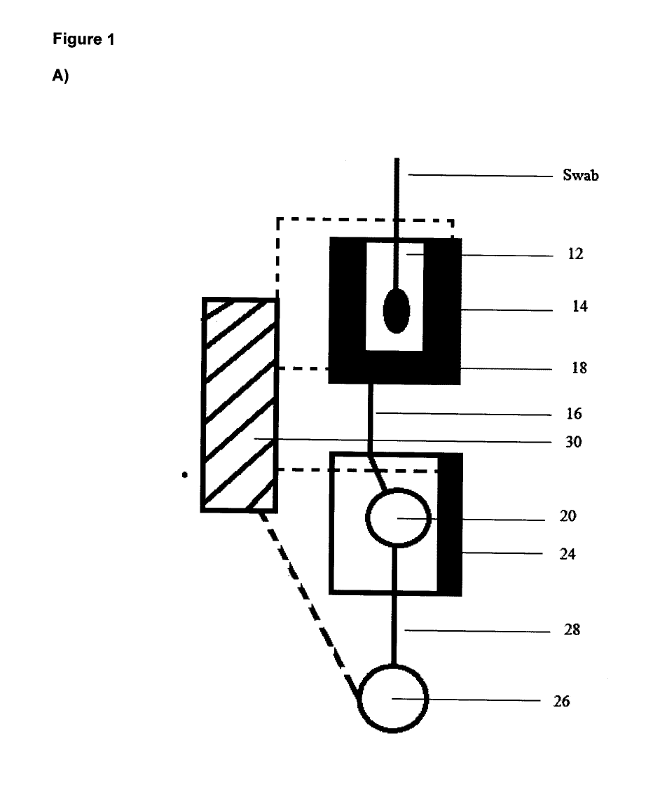

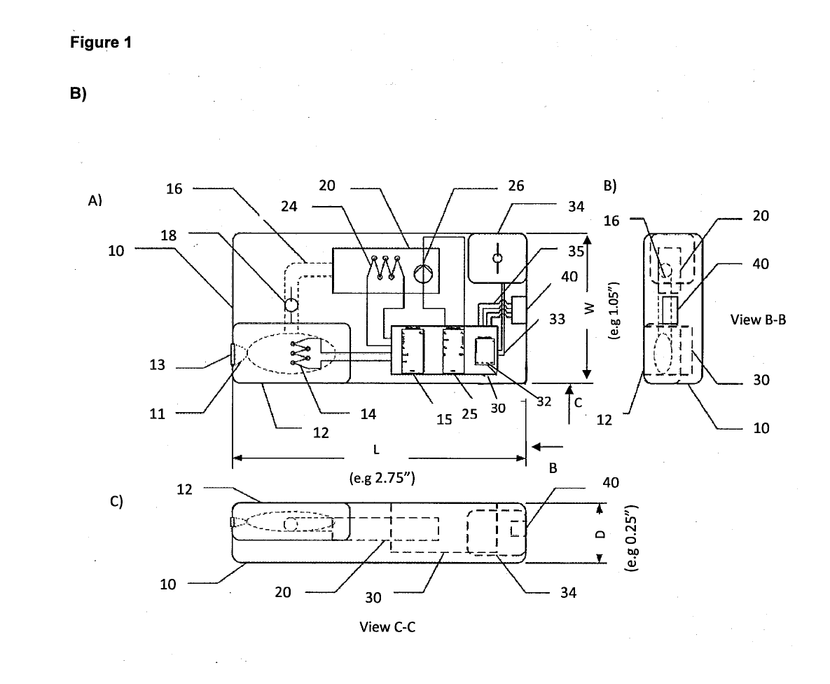

A hand-held, disposable device 10 is provided (FIG. 1B). The device 10 comprises a first extraction chamber 12 having a maximum volume of about 250 μl. The extraction chamber 12 includes a lysing reagent of PBS with 0.1% Triton X-100. The extraction chamber 12 includes an opening 11 for accepting the head of a sample-containing swab and the chamber 12 is sized to accept the swab. A lid 13 is provided to seal opening 11 of the extraction chamber 12. Closing of lid 13 activates the device 10 by causing release of buffer into the extraction chamber 12 from a blister pack. The extraction chamber 12 is fitted with a first self-regulating heater 14, activated on closing lid 13, that heats the extraction chamber 12 to a temperature of about 95° C. and maintains this temperature for at least 2 minutes, e.g. by the use of a timer. The heater 14 is connected to control unit 30 and powered by battery 34.

The extraction chamber 12 is connected by a microfluidic channel 16 to an amplification chamber 20. A pump means 18, e.g. a micropump or syringe, is located within the microfluidic channel 16 and functions to move lysed material from the extraction chamber 12 to the amplification chamber 20. The pump 18 is connected to control unit 30 and powered by battery 34. The control unit also includes a D/A converter 15 for temperature and/or potential controls, and an A/D converter 25 to convert analogue detection signals to digital signals. A timer activates the pump 18 at the appropriate time. The amplification chamber 20 includes a second self-regulating heater 22, activated by a timer as described, which maintains the temperature within the amplification chamber 20 at 62° C. for 20 minutes to allow for isothermal amplification, including loop-mediated isothermal amplification (LAMP), cross-priming amplification (CPA), recombinase polymerase amplification (RPA), rolling circle amplification (RCA), helicase-dependent amplification (HDA), single-mediated amplification of RNA technology (SMART), nicking enzyme-mediated amplification (NEMA), isothermal chain amplification (ICA), Smart amplification (Smart-AMP), exponential amplification reaction (EXPAR), ramification amplification (RAM), or nicking end amplification reaction (NEAR).

Once the lysed material is cooled to the target temperature of about 62° C. over time, between 5 and 10 μl of lysed material is transferred to the amplification chamber 20 by pump 18. The lysed material is maintained at about 62° C. with a second self-regulating heater 24 within the amplification chamber. The second heater 24 is connected to control unit 30 and powered by battery 34. The amplification chamber contains an amplification master mixture of salt buffer, DNA polymerase, 5 mM MgSO₄, and target-specific primers, e.g. 15 μl of salt buffer solution, 1 μl of polymerase, and 4 μl of specific primers, with 5 to 10 μl of DNA binding dye (Quant-iT PicoGreen, hydroxynapthol blue, and leuco triphenylmethane dye).

The amplification chamber 20 contains a detection sensor 26, which could be a colorimeter for RGB analysis, a fluorimeter to detect fluorescence changes after amplification with a fluorescent dye, a potentiometer to perform electrochemical analysis of the sample with methylene blue, or a clear viewing port to analyze a visual colour change.

In an alternate embodiment illustrated in FIG. 1A, the amplification chamber 20 is connected to a separate detection chamber 50, via a microfluidic channel 28, and the detection chamber 50 includes the detection sensor 26.

### Lysis Validation

To confirm that the extraction chamber could reach a target temperature (93° C.) using a self-regulating heater and maintain this temperature for two minutes, the temperature was monitored for 20 minutes using a thermocouple. Different resistances (3.8 ohm, 3.5 ohm, and 3.4 ohm) were tested. All measurements were performed in a tinfoil chamber attached to the PTC heater coupled with a thermal paste. To further confirm that these conditions were sufficient to lyse various clinical samples, swabs containing Respiratory Syncytial Virus A (RSV-A), Streptococcus agalactiae, and Influenza virus H1 were each placed within the extraction chamber containing 250 μl of PBS with 0.1% Triton-X100 and lysed for 3 minutes followed by amplification using the amplification mix, Optigene Lamp mastermix, on the fluorimeter, the Genie II instrument. Amplification times were compared with an unlysed control in each case. Oligonucleotide primers from Table 1 used in each case were as follows: primers for RSV-A having SEQ ID NOs: 20, 21, 22, 23 and 24), primers for Streptococcus agalactiae having sequences of SEQ ID NOs: 32, 33, 34, 35, and 36, and primers for Influenza virus H1 having sequences of SEQ ID NOs: 7, 8, 9, 10 and 11.

### Amplification Validation

To confirm that the amplification chamber 20 reached the required temperature (62° C.) and maintained this temperature for 20 minutes, a thermistor was used to monitor the chamber 20 temperature over time. Using a constant resistance of 8.6 ohm, the temperature was monitored for 20 minutes. To further confirm that the samples were successfully amplified under these conditions, RSV-A, S. agalactiae, and influenza virus H1 were heat-lysed on a heat-block at 95° C. for 10 minutes and amplified in the amplification chamber 20 containing 15 μl of Optigene mastermix and 5 μl of specific primers as described above for 15 minutes. The sample was then removed from the amplification chamber, mixed with 10 μl of SYBR Green DNA binding dye, and end-point fluorescence at between 500 and 520 nm was determined on the Genie II instrument compared to an unamplified negative control.

### Detection

To evaluate whether amplified DNA could be detected either visually or using electrochemical detection, a variety of DNA binding dyes including Quant-iT PicoGreen (Life Technologies), hydroxynapthol blue, leuco triphenylmethane dye, and methylene blue were used. Approximately 100 copies of either RSV-A, S. agalactiae, or influenza virus H1 were amplified using the amplification mixture including primers as described above, Optigene LAMP mastermix, after which the amplified material was mixed with 10 μl of individual DNA binding dyes for 3 minutes at room temperature. For Quant-iT PicoGreen, hydroxynapthol blue, and leuco triphenylmethane dye, a visual change in the colour of the dye was observed by at least 10 individuals. For methylene blue, detection was achieved based on changes in the peak anodic current in the sample using cyclic voltammetry with a PalmSens potentiostat and compared to a baseline reading prior to amplification.

### Sample Analysis

Approximately 500 S. agalactiae cells, RSV-A particles, or influenza particles H1 were applied to respective nasopharyngeal swabs to mimic a clinical swab sample from an infected patient. The swab was placed in the extraction or extraction chamber 12 of a device 10. The extraction chamber 12 contained 250 μl of phosphate buffer saline and 0.1% Triton X-100, which was subsequently heated to 95° C. using a self-regulated heating device. This temperature was maintained for 3 minutes, after which 25 μl of the lysed solution was transferred to the amplification chamber 22 of the device 10 using a pipette. The 25 μl of lysed solution was mixed with LAMP amplification buffer containing a strand-displacement DNA polymerase from Geobacillus, primers (5 μl total) targeting a specific S. agalactiae, RSV-A, or influenza H1 gene (as described above), 5 μl of dNTPs, 1 μl of MgSO₄, and 15 μl of a salt-buffer at pH 9.2. The amplification chamber 22 was maintained at 63° C. using a self-regulated heater for 20 minutes to allow amplification to occur. Amplification was monitored using a fluorescent dye (10 μl of SYBR green), and amplification was detectable within 14 minutes. Total time to detect S. epidermidis from a clinical swab was 19 minutes, which included a 5 minute sample release and lysis step.

Electrochemical detection of DNA amplification was also used. After incubation for 20 minutes at 63° C. in the amplification chamber 22, the sample was analyzed using methylene blue detection and cyclic voltammetry with a PalmSens potentiostat and compared to a baseline reading taken prior to amplification. Decrease in peak anodic current is indicative of amplification. Methylene blue (MB) at two different concentrations was used to detect 500 ng of amplified DNA. Methylene blue alone was used as a control.

### Results

### Analysis of Self-Regulated Heating

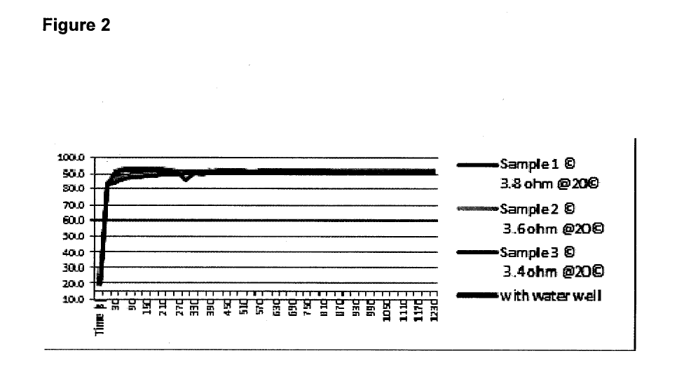

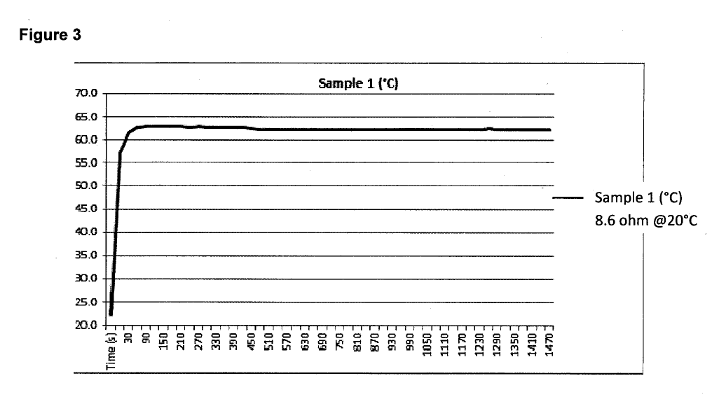

Accurate temperature control was confirmed in the device 10 for lysis of infectious materials and for amplification of DNA. Self-regulating heaters to maintain temperature in the extraction chamber at 93° C. and in the amplification chamber at 62° C. were used. For the lysis temperature, various resistances were tested to explore whether the self-regulating heater could function appropriately under various conditions at room-temperature. It was found that the temperature of 93° C. was maintained for over 20 minutes at all resistances tested (FIG. 2). For the amplification temperature, a temperature of 62° C. could be maintained at 8.6 ohm for 20 minutes (FIG. 3). Based on these results, the self-regulating heaters were determined to be sufficient to maintain temperatures for lysis and amplification.

### Evaluation of Sample Lysis

To confirm that the device 10 was sufficient to lyse clinical material and sterilize clinical specimens within 3 minutes at 93° C., Respiratory Syncytial Virus (RSV), Influenza, E. coli, and S. pneumoniae were separately introduced into the extraction chamber 12 of the device 10 for subsequent analysis. It was first determined that virus and bacteria were killed in the extraction chamber 12, to ensure that the device is not a biohazard after being used. To accomplish this, 10,000 bacterial or viral particles were introduced into the extraction chamber, were lysed and then were analyzed either based on a plating assay (for bacteria) or through infection and cell culture (for viruses). No infectious particles remained after exposure to the extraction chamber 12, indicating that exposure to 93° C. for 3 minutes kills 100% of the virus or bacteria. It was then determined that these conditions released intracellular DNA and RNA that could be used for DNA amplification. Following lysis of each sample, the resulting DNA or RNA was analyzed using LAMP on a Genie II instrument compared to a positive lysis control (bead lysis). It was found that LAMP could detect released nucleic acid from all four specimens, suggesting that the extraction chamber could be used to release nucleic acid for subsequent analysis.

### Evaluation of Isothermal DNA Amplification In Situ

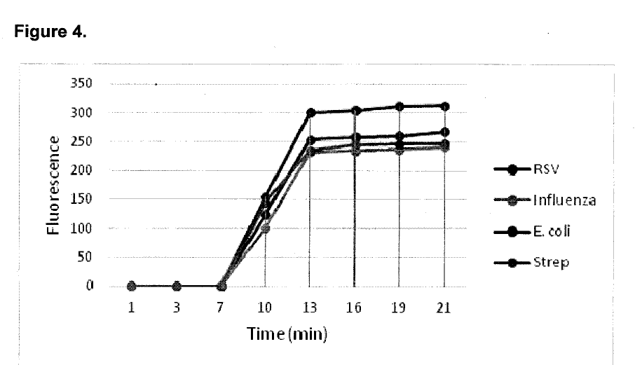

To confirm that isothermal amplification could be performed in the device 10, RSV, Influenza, E. coli, and S. pneumoniae samples were bead lysed and amplified within the the amplification chamber 22. The samples were subsequently analyzed based on end-point fluorescence with Sybr Green DNA binding dye. All specimens were amplified in the chamber within 10 minutes (FIG. 4).

### DNA Detection In Situ

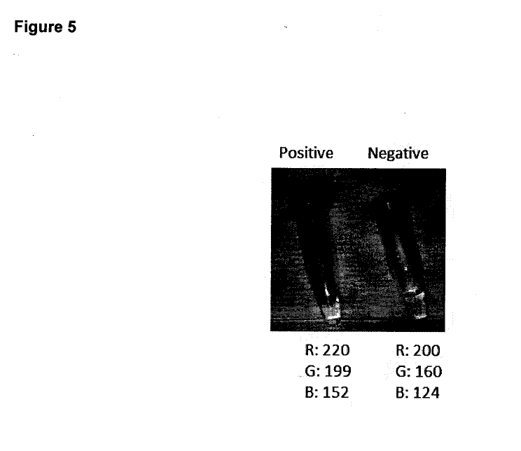

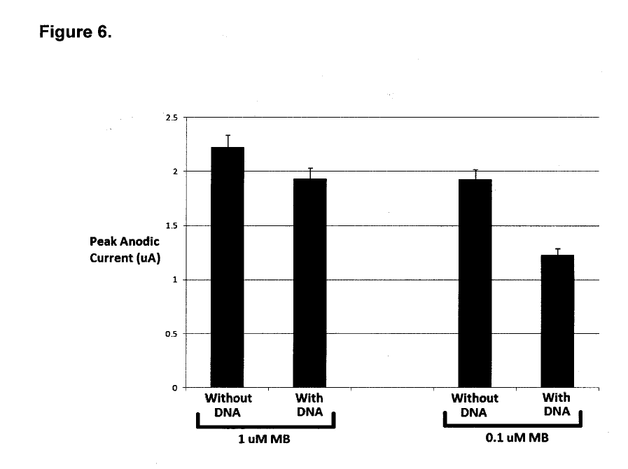

Detection of DNA was performed using both a visual color using Quant-It PicoGreen DNA binding dye and through potentiometry using Methylene Blue. To accomplish this, amplified DNA was mixed with either Quant-It PicoGreen or Methylene Blue dye and analyzed either visually or using potentiometry, respectively. For visual detection, color change in the presence of DNA, and lack of color change in the absence of DNA, was detectable by the naked eye (FIG. 5). For methylene blue (MB) DNA detection, peak anodic current in the presence and absence of DNA was determined. A significant decrease in the peak anodic current of 20 to 25% was observed in the presence of DNA, which was indicative of DNA amplification (FIG. 6). At lower MB concentrations (0.1 μM), there was a greater decrease in peak anodic current compared to higher MB concentrations (1 μM) in the presence of DNA.

### Clinical Analysis

The device 10 was tested using clinical Influenza nasopharyngeal swabs, RSV nasopharyngeal swabs, and Streptococcus throat swabs. Swabs were obtained from infected patients that were known to be positive based on culturing methods. The swabs were then inserted into the extraction chamber 12 of the device 10. The extraction chamber was filled with 250 μl of PBS and heated at 93° C. for 3 minutes. Subsequently, the lysed material was pumped into the amplification chamber 22, where it was mixed with LAMP mastermix. The amplification chamber 22 was then activated for 15 minutes (heated to 62° C.) and DNA was detected visually after mixing with Quant-It PicoGreen DNA binding dye in the viewing vials. Infectious material was detected within 20 minutes using the device.

TABLE 1
Oligonucleotide primers for isothermal amplification and detection of various
viral and bacterial pathogens

| | Gene | Amplification | |
|---|---|---|---|
| Organism | target | method | Primers (5′ to 3′) |
| Influenza A | Matrix | LAMP | AGGATGGGGGCTGTAACC (SEQ ID NO: 1) |
| H3 | | | CCAGCCATTTGCTCCATAGC (SEQ ID NO: 2) |
| | | | TGAGACCTGTGCTGGGAGTCAAGGTGGCATTGGCCTGGTA (SEQ ID |
| | | | NO: 3) |
| | | | TAGGCAGATGGTGGCAACAACCTGTAGTGCTGGCCAAAACC (SEQ |
| | | | ID NO: 4) |
| | | | AATCTGCTCACATGTTGCACA (SEQ ID NO: 5) |
| | | | CATTAATAAAACATGAGAACAGAAT (SEQ ID NO: 6) |
| Influenza A | Matrix | LAMP | CCGTTTTACTCGTGCCGC (SEQ ID NO: 7) |
| H1 | | | |
| | | | AGACGCTTTGTCCAAAATGC (SEQ ID NO: 8) |
| | | | TCACAAGTGGCACACACTAG (SEQ ID NO: 9) |
| | | | CCTMGCCCCATGGAACGTTATGGGGACCCGAACAACATG (SEQ ID |
| | | | NO: 10) |
| | | | TTCAACTGGTGCACTTGCCAGTGTGGTCACTGTTCCCATCC (SEQ ID |
| | | | NO: 11) |
| | | | TGAGCTTCTTGTATAGTTTAACTGC (SEQ ID NO: 12) |
| | | | TGCATGGGCCTCATATACAACA (SEQ ID NO: 13) |
| Influenza B | NS1 gene | LAMP | AGGGACATGAACAACAAAGA (SEQ ID NO: 14) |
| | | | CAAGTTTAGCAACAAGCCT (SEQ ID NO: 15) |
| | | | TCAGGGACAATACATTACGCATATCGATAAAGGAGGAAGTAAAC |
| | | | ACTCA (SEQ ID NO: 16) |
| | | | TAAACGGAACATTCCTCAAACACCACTCTGGTCATAGGCATTC |
| | | | (SEQ ID NO: 17) |
| | | | TCAAACGGAACTTCCCTTCTTTC (SEQ ID NO: 18) |
| | | | GGATACAAGTCCTTATCAACTCTGC (SEQ ID NO: 19) |
| RSV A | Matrix | LAMP | GCTGTTCAATACAATGTCCTAGA (SEQ ID NO: 20) |
| | | | GGTAAATTTGCTGGGCATT (SEQ ID NO: 21) |
| | | | TCTGCTGGCATGGATGATTGGAGACGATGATCCTGCATCA (SEQ ID |
| | | | NO: 22) |
| | | | CTAGTGAAACAAATATCCACACCCAGCACTGCACTTCTTGAGTT |
| | | | (SEQ ID NO: 23) |
| | | | ACATGGGCACCATAT'TGTAAG (SEQ ID NO: 24) |
| | | | AGGGACCTTCATTAAGAGTCATGAT (SEQ ID NO: 25) |
| RSV B | Pol gene | LAMP | AACCATTCCTGCTACAGAT (SEQ ID NO: 26) |
| | | | CATCTTGAGCATGATATTTTGC (SEQ ID NO: 27) |
| | | | AGCATCGCAGACAAAGATACTAATCAACTAACAACATACATTGG |
| | | | TCT (SEQ ID NO: 28) |
| | | | CCTGTCACAGCCAATTGGAGTCAGAAGAACAGTATTTGCACTT |
| | | | (SEQ ID NO: 29) |
| | | | AACGCCGTCAACGACGTCGTGCCCTCGAGGACCTGCTC (SEQ ID |
| | | | NO: 30) |
| | | | AGGTTCTGCAAATTTTATATGTAAATA (SEQ ID NO: 31) |
| S. agalactiae | sobA gene | LAMP | AGGCGCTCTTAGCTGATGT (SEQ ID NO: 32) |
| | | | TGCATGGTGCTTATCATGATGT (SEQ ID NO: 33) |
| | | | ACCACCGTTATTGATGACTG (SEQ ID NO: 34) |
| | | | ATATGATGCGCTTGAGCC (SEQ ID NO: 35) |
| | | | ACATCCTGAAATTGGAGAAGACTTTTTTCCTGACGAATATCTTCTGGAA |
| | | | T (SEQ ID NO: 36) |
| | | | GAGCAGCATTTGCATTAGCAACATATTTTGATGCTGAGACAATGACAC |
| | | | (SEQ ID NO: 37) |
| S. aureus | Nuc gene | LAMP | CAAACCTAACAATACACATGAACA (SEQ ID NO: 38) |
| | | | ACGCTAAGCCACGTCCATAT (SEQ ID NO: 39) |
| | | | CGTTGTCTTCGCTCCAAAT (SEQ ID NO: 40) |
| | | | TGCAAAGAAAATTGAAGTCGA (SEQ ID NO: 41) |
| | | | TCAAGGCTTGGCTAAAGTTGCTTATTCGCTTGTGCTTCACTT (SEQ ID NO: |
| | | | 42) |
| | | | CGTTTACCATTTTTCCATCAGCATATTTGACAAAGGTCAAAGAACT (SEQ |
| | | | ID NO: 43) |
| HSV1 | UL3 gene | EXPAR | CTGGCGATAT (SEQ ID NO: 44) |
| | | | ATATCGCCAG (SEQ ID NO: 45) |
| | | | ATATCGCCAGGTGAGACTCTATATCGCCAG (SEQ ID NO: 46) |
| | | | CTGGCGCTTGATGGTATCCAGACTCTATATCGCCAG (SEQ ID NO: 47) |
| M. | gyrB | EXPAR | GAGTCCAGTATTTGGTCGTCTGTCCTGCGTAGCGACTC(SEQ ID NO: |
| tuberculosis | | | 48) |
| | | | ATTTGGTCGTCGCAGACTCATTTGGTCGT (SEQ ID NO: 49) |
| | | | ACCGGGCAGATTCGGCCCACTTCCCGCAGACTCATTTGGTCGT |
| | | | (SEQ ID NO: 50) |
| | | | TTTTTTTTTACCGGGCAGATT (SEQ ID NO: 51) |
| | | | CGGCCCACTTCCTTTTTTTTT (SEQ ID NO: 52) |
| | | | biotin-sp18-AATCTGCCCGGTAAAA (SEQ ID NO: 53) |
| Influenza H5 | HA gene | SMART | TCAAGAGTAGACACAGGATCAGCATAGGCAATAGATGGAGTCAC |
| | | | GTAATCAGATCAGAGCAATAGGTCA (SEQ ID NO: 54) |
| | | | ATGGTAGATGGTTGGTATGGGTA (SEQ ID NO: 55) |
| | | | CGTAGGCAATAGATGGAGTCACTACG(SEQ ID NO: 56) |
| | | | AATIVTAATACGACTCACTATAGGGAGAAGGTGACCTATTGCTCT |
| | | | GATCTGATTAC (SEQ ID NO: 57) |
| | | | TAATACGACTCACTATAGGTGACCTATTGCTCTGATCTGATTAC |
| | | | TCAAGAGTAGACACAGGATCAGCAT (SEQ ID NO: 58) |
| Influenza H5 | HA gene | RCA | (PLP)GGATGATCTGANITTTCTCAAACCCGOTCAACTTCAAGCTC |
| | | | CTAAGCCTTGACGAA (SEQ ID NO: 59) |
| | | | GCTTAGGAGCTTGAAGTTG*A*C (SEQ ID NO: 60) |
| | | | GCTTTGCCTGACTGAATGC*A*G (SEQ ID NO: 61) |
| H. pylori | ureC | HAD | CTTTTAGGGGTGTTAGGGGT (SEQ ID NO: 62) |
| | | | AAGCTTACTTTCTAACACTAACGC (SEQ ID NO: 63) |
| | | | CGATTGGGGATAAGTTTTGTG (SEQ ID NO: 64) |
| S. aureus | mecA | RPA | TCCAACATGAAGATGGCTATCGTGTCACAATCGTT (SEQ ID NO: 65) |
| | | | CCTGTTTGAGGGTGGATAGCAGTACCTGAGCC (SEQ ID NO: 66) |
| S. enterica | invA | RPA | TACCGGGCATACCATCCAGAGAAAATCGGGCCGC (SEQ ID NO: 67) |
| | | | ATTGGCGATAGCCTGGCGGTGGGTTTTGTTGT (SEQ ID NO: 68) |
| S. | gseA | LAMP | AACATCACTGTTACTGGTTAC (SEQ ID NO: 69) |
| epidermderis | | | CTGCTATTGTATTTATTATCTACGC (SEQ ID NO: 70) |
| | | | CTCGCCACCAATATAGACAACTTTTGGTGACAAACCATTAGCC |
| | | | (SEQ ID NO: 71) |
| | | | GACCTAAGTACTGTAGGTGGAAACTCACCATAATGTATTCCAAT |
| | | | AACTTG (SEQ ID NO: 72) |

Primers exemplified in Table 1 have been used in accordance with the following references which are incorporated herein by reference:
[1] Mahony J. et al. 2013; J Clin Viral 2013 58:127-131.
[2] Mahony J. et al. 2013; J. Clin. Microbiol. 2013 doi:10.1128/JCM.00662-13.
[3] Deguo Wang and Yanhong Liu Int. J. Environ, Res. Public Health 2015, 12, 5735-5742; doi:10.3390/ijerph120605735
[4] Wang D. et al. Molecules 2015 20, 9487-9495
[5] Tan E. et al. 2007; Clin Chem 53(11) 2017-2020.
[6] Roskos K. et al. 2013; PLOS One 8(7):e69355.
[7] McCalla et al. 2012; J Molec Diagn 14(4):328-335.
[8] Hamidi S. et al. 2015; Analyst 140, 1502-1509.
[9] Gill P. et al. 2007; Diagn Microbiol Infect Dis 59:243-249.
[10] Kersting S. et al. 2014; Microchim Acta 181:1715-1723.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description has been presented for purposes of illustration and description, but is not intended to be exhaustive or limited to the form disclosed. Many modifications and variations will be apparent to those of ordinary skill in the art without departing from the scope of the claims. The embodiment was chosen and described in order to best explain the principles of the technology and the practical application, and to enable others of ordinary skill in the art to understand the technology for various embodiments with various modifications as are suited to the particular use contemplated.

One or more currently preferred embodiments have been described by way of example. It will be apparent to persons skilled in the art that a number of variations and modifications can be made without departing from the scope of the claims.

What is claimed is:

1. A point-of-care device for detecting a target nucleic acid comprising:
an extraction chamber adapted to receive a biological sample, wherein said extraction chamber comprises means to extract and lyse the sample to release nucleic acid;
a first amplification chamber in communication with the extraction chamber, wherein said amplification chamber comprises means to trigger nucleic acid amplification of a target nucleic acid sequence to occur; and
a detection chamber in communication with the amplification chamber, wherein said detection chamber comprises means to detectably label the target nucleic acid and means to detect a signal associated with labeled target nucleic acid.

2. The device of claim 1, which additionally comprises a processing device adapted to receive the signal associated with labeled target nucleic acid and provide an output that indicates the presence or absence of labeled target nucleic acid, or means to connect to a processing device adapted to receive the signal associated with labeled target nucleic acid and provide an output that indicates the presence or absence of labeled target nucleic acid.

3. The device of claim 1, wherein the extraction chamber comprises a lysis solution and a heater to extract and lyse the sample.

4. The device of claim 1, wherein the amplification chamber comprises an amplification mixture comprising oligonucleotide primers for amplification of the target nucleic acid sequence, a DNA polymerase, deoxynucleoside triphosphates, buffer and magnesium, and a heater to trigger nucleic acid amplification.

5. The device of claim 1, wherein the detection chamber comprises a detectable label for labeling the target nucleic acid and a detection sensor suitable to detect labeled target nucleic acid.

6. The device of claim 5, wherein the detectable label is selected from the group consisting of fluorescent labels, chemiluminescent labels, chromogenic labels, and electrochemically detectable labels.

7. The device of claim 1, wherein the extraction chamber, amplification chamber and detection chamber are a single chamber.

8. The device of claim 1, wherein the detection chamber is within the amplification chamber.

9. The device of claim 1, wherein the device comprises a second amplification chamber in communication with the extraction chamber adapted to amplify a positive control nucleic acid sequence, wherein said second amplification chamber comprises an amplification mixture comprising oligonucleotide primers complementary to a control nucleic acid sequence.

10. The device of claim 1, wherein the device comprises 2 or more amplification chambers in communication with the extraction chamber, wherein each amplification chamber is adapted to amplify a target nucleic acid sequence from a different target microorganism.

11. The device of claim 10, wherein each amplification chamber comprises amplification mix comprising oligonucleotide primers complementary to target nucleic acid sequence from a different target microorganism.

12. The device of claim 1, comprising a pumping means to transfer released nucleic acid to the amplification chamber.

13. The device of claim 1, which is disposable.

14. The device of claim 1, which is hand-held.

15. A method of detecting target nucleic acid in a biological sample is provided comprising:
collecting a biological sample on a swab and inserting the swab in a contained extraction chamber adapted to extract the sample from the swab and lyse pathogen to release nucleic acid;
transferring a portion of the released nucleic acid to a nucleic acid amplification chamber connected to the extraction chamber and exposing the nucleic acid to reagents to induce nucleic acid amplification;
label and detect target nucleic acid in a detection chamber connected to the amplification chamber; and
obtain an output from the detection chamber which indicates the presence or absence of the target nucleic acid.

16. The method of claim 15, wherein the extraction chamber is heated for pathogen lysis and release of nucleic acids.

17. The method of claim 15, wherein the extraction chamber is lined with target-capture DNA probes immobilized on the chamber wall.

18. The method of claim 15, wherein a positively charged electric field is utilized within the extraction chamber to accelerate binding of nucleic acid to the target capture probes.

19. The method of claim 15, wherein a motor is used to facilitate mechanical lysis and release of pathogens in the extraction chamber.

20. The method of claim 15, wherein a heater is used to maintain a temperature of 58° C. to 66° C. in the nucleic acid amplification chamber.

21. The method of claim 15, whereby isothermal DNA amplification is used to amplify specific pathogenic sequences.

22. The method of claim 15, where electrochemical detection of DNA is accomplished using methylene blue DNA binding dye.

23. The method of claim 15, where DNA is detected using a lateral flow assay.

24. The method of claim 15, where diagnostic information is transmitted to an on-board reader.

25. The method of claim 15, where diagnostic information is transmitted to a cell phone, laptop or tablet through a USB connection or by directly capturing a graphic image.

26. The method of claim 15, where the diagnostic platform is powered through a USB connection from a cell phone or laptop.
